(19) United States
(12) Patent Application Publication
(10) Pub. No.: US 2026/0306164 A1
(43) Pub. Date: Oct. 8, 2026

(54) HF INSTRUMENT HAVING A JAW PART

(71) Applicant: Aesculap AG, Tuttlingen (DE)

(72) Inventor: Tom Schweitzer, Tuttlingen (DE)

(21) Appl. No.: 19/476,278

(22) PCT Filed: Apr. 12, 2024
(86) PCT No.: PCT/EP2024/059961

§ 371 Date: Oct. 17, 2025

(30) Foreign Application Priority Data
Apr. 19, 2023 (DE) 10 2023 109 870.5

(51) Int. Cl. A61B 18/14; A61B 18/00

(52) U.S. Cl. CPC A61B 18/1445; A61B 2018/00184; A61B 2018/00601; A61B 2018/0063; A61B 2018/1455

(57) ABSTRACT

An HF instrument, in particular a bipolar instrument for sealing vessels, includes at least one metal electrode with a contact surface for contacting tissue. The metal electrode includes a support portion that faces away from the contact surface and has or is a porous support structure. The metal electrode can be produced using a generative manufacturing process.

### CROSS-REFERENCE TO RELATED APPLICATIONS

This application is the United States national phase entry of International Application No. PCT/EP2024/059961, filed on Apr. 12, 2024, and claims the benefit of priority of German Application No. 10 2023 109 870.5, filed on Apr. 19, 2023.

### FIELD

The present disclosure relates to a high-frequency medical surgical instrument (HF instrument), in particular a bipolar vessel sealing instrument, having at least one metal electrode in a jaw part.

### BACKGROUND

In high-frequency surgery (hereinafter referred to as HF surgery), a high-frequency alternating current is passed through the human body or a part of the body in order to selectively cauterize (coagulation) or cut (electrotomy) tissue by the heating caused thereby. The damaged tissue is later absorbed by the surrounding healthy tissue. A significant advantage over conventional cutting techniques using a scalpel is that bleeding may be stopped at the same time as the cut by closing the affected vessels, in the sense of coagulation.

Currently, the monopolar HF technique is most commonly used in HF surgery. Here, one pole of the HF voltage source is connected to the patient via a counter electrode with the largest possible surface area, for example through contacts on the operating table on which the patient is lying, through contact armbands or contact foot bands or through adhesive electrodes. This counter electrode is often called a dispersing electrode or neutral electrode. The other pole is connected to the surgical instrument and this forms the so-called active electrode or working electrode. The current flows via the path of least resistance from the active electrode to the neutral electrode. The current density is highest in the immediate vicinity of the active electrode, where the thermal effect is strongest. The current density decreases with the square of the distance. The neutral electrode should have as large an area as possible and is to be well connected to the body so that the current density in the body is kept low and no burns occur. The skin on the neutral electrode is not noticeably heated due to the large surface area. Strict safety measures apply when attaching the neutral electrode. Correct positioning and good contact of the neutral electrode (depending on the surgical site) are crucial to avoid burns.

With the bipolar HF technique, in contrast to the monopolar technique, the current flows through a small part of the body—the part in which the surgical effect (incision or coagulation) is desired. Two metal electrodes insulated from each other, which are held in jaw parts of the HF instrument and between which the HF voltage is applied, are guided directly to the surgical site. The circuit is closed via the tissue between them. The thermal effect takes place in the tissue between the metal electrodes.

In such HF instruments, in particular bipolar sealing instruments, the jaw parts are preferably manufactured/configured in a sandwich construction. The jaw parts consist of or have the thin metal electrode, which acts as a contact surface to the tissue, a plastic spacer, which provides electrical and thermal insulation, and a carrier component, which is provided and configured to ensure force transmission and contains a closing mechanism. The carrier component provides the jaw part with the necessary stability and rigidity.

However, such a jaw part configured in a sandwich construction has a complex and cost-intensive manufacturing process. The various components that have to be joined together result in an accumulation of manufacturing tolerances, which reduces the accuracy of fit and therefore the quality of the jaw part or HF instrument.

One approach to eliminating these disadvantages is to configure/form the metal electrode or a branch of the jaw part as a solid component, i.e. the metal electrode is manufactured entirely from a (non-porous) solid material according to this prior art.

Such an overall solid metal electrode is particularly rigid against thermal and/or mechanical deformation, but has a high thermal mass, which in particular with larger jaw parts leads to the fact that a large part of the energy supplied to the metal electrode by an HF generator does not flow into tissue sealing, but into heating of the solid metal electrode.

### SUMMARY

The object of the present disclosure is to eliminate or at least reduce the disadvantages of the prior art. Specifically, the object of the present disclosure is to provide an HF instrument with a jaw part that is easy to manufacture, has low tolerances, and a low thermal mass.

Advantageous Developments Are Described Below.

Specifically, the object is solved by an HF instrument, in particular a bipolar vessel sealing instrument, with at least one metal electrode which comprises a solidly configured, i.e.

non-porous or closed contact surface for contacting tissue. The metal electrode further comprises a support portion facing away from the contact surface and which has or is a porous or open support structure. Accordingly, one aspect of the present disclosure is to form/provide (preferably exclusively) the electrode side to be brought into (physical) abutment/contact with a patient tissue completely or in sections as a closed, non-porous contact surface, for example by arranging a (thin) closed metal platelet, a closed metal layer, a closed metal coating, etc., and to form/provide the remaining metal electrode at least partially or completely as or with an open (porous or lattice-like, etc.) support structure, which is provided and configured to support the contact surface (at least in certain areas) and to give it stability against load-induced (mechanical and/or thermal) deformation.

Solid means that the contact surface is uninterrupted and smooth.

In other words, the object is solved by the HF instrument, which is configured, for example, as a preferably bipolar HF instrument in a scissor or pincer configuration or a laparoscope configuration. The HF instrument comprises at least one, preferably two, metal electrode(s) configured in a jaw part of the HF instrument in a respective branch.

The preferably two branches are movable relative to each other via an articulation or hinge. The metal electrode includes at least one flat portion, which forms the contact surface and faces a clearance/interior space of the jaw part, i.e. forms a front side of the metal electrode. The contact surface is provided and configured to contact the tissue. The metal electrode further comprises the support portion, which is configured/oriented away from the clearance of the jaw part, i.e. forms a rear side of the metal electrode. The support portion includes or is the porous support structure.

In yet other words, the HF instrument in particular has a distal instrument on which the metal electrode is arranged. In particular, the distal instrument has a jaw part with two branches, at least one of which is pivotable toward the respective other branch, wherein the metal electrode is arranged on at least one of the branches on a side facing the respective other branch. Further preferably, the HF instrument has a proximal handle and a shaft connecting the handle to the distal instrument.

The porous support structure is preferably configured from metal. Porosity means that the support structure has a proportion of hollow space, preferably configured uniformly over its volume. In other words, the support structure is configured with a plurality of three-dimensionally arranged hollow spaces.

Such a porous support structure allows the metal electrode to be stiffened without significantly increasing the thermally relevant mass of the metal electrode. Furthermore, the open-pored configuration of the support structure enables good air and gas circulation, which improves thermal insulation.

The porous support structure may be an additively manufactured structure. Additive manufacturing is a manufacturing method that is configured by material deposition, preferably in layer build-up processes, and thus differs from abrasive manufacturing methods in which material is removed.

In other words, the porous support structure may be a 3D-printed structure, which may be configured, for example, by selective laser melting, selective electron beam melting, laser deposition welding, wire arc/plasma arc energy deposition or wire feed electron deposition.

In one aspect, the metal electrode may be monolithically configured with the support portion. Monolithic is to be understood as consisting of a uniform, non-separable unit.

In other words, the metal electrode may be configured in one piece of material. This means that at least the contact surface and the support portion with the porous support structure may be configured monolithically/in one piece of material.

Due to the monolithic configuration of the metal electrode, a high component rigidity may be achieved. Furthermore, the manufacture of the metal electrode may be simplified, as there is no need for a joining process between the support portion and the contact surface. In addition, the reduction of the components may prevent the accumulation of manufacturing tolerances, which increases the manufacturing accuracy and thus the quality of the jaw part of the HF instrument.

In a further aspect, the metal electrode may comprise a blade-guiding channel for guiding a blade, which is open toward the contact surface and extends in a longitudinal direction of the metal electrode.

In other words, the metal electrode may be integrally configured with the blade-guiding channel. The blade-guiding channel may have a substantially U-shaped cross-sectional area, wherein the blade-guiding channel is open toward the clearance of the component and closed away from the clearance. In a state in which the jaw part is closed, i.e. the contact surfaces of the two branches are aligned in parallel, the blade-guiding channels of the two metal electrodes may face each other with their respective openings (i.e. may together define a space with a preferably rectangular or oval cross-section).

The metal electrode may have an elongate shape and the blade-guiding channel may extend in the longitudinal direction of the metal electrode. With respect to a width direction of the metal electrode oriented transversely to the longitudinal direction, the blade-guiding channel may be arranged centrally or at least substantially centrally.

In yet other words, the blade-guiding channel may be configured as a groove-shaped geometry in the contact surface.

By configuring the metal electrode with the blade-guiding channel, the number of components of the branch and thus the number of components of the jaw part may be further reduced.

In a further aspect, the blade-guiding channel and the contact surface may be configured substantially T-shaped with each other and the porous support structure may be configured in a space or portion delimited by the contact surface and the blade-guiding channel.

In other words, the blade-guiding channel may extend at the rear side of the contact surface oriented normal to the contact surface in a kind of stepped shape and the blade-guiding channel with the contact surface may delimit at least one, in particular two, volumes/spaces positioned next to the blade-guiding channel. The porous support structure may be configured in this space/in these spaces. The porous support structure may be connected/configured with the contact surface and the blade-guiding channel, preferably in one piece of material.

With such a configuration, the metal electrode may be compact, light and stable.

The blade-guiding channel may have a closed geometry toward the rear side.

In a further aspect, the support portion may include solid (in the sense of closed/non-porous) stiffening ribs.

In other words, in addition to the porous support structure, the support portion may include the solid stiffening ribs, which preferably segment the support portion, and the porous support structure is arranged/configured in the thus configured segments.

In this way, the porous support structure may be configured more easily and is less susceptible to defects, in particular to manufacturing defects such as local layer bonding defects/ defects in the additively manufactured porous support structure.

In a further aspect, the contact surface of the metal electrode may be configured to be tongue-shaped, wherein the stiffening ribs are aligned normal to the blade-guiding channel and may preferably be arranged/configured substantially uniformly or sectionally uniformly distributed over a longitudinal extension of the metal electrode.

In other words, the contact surface may be a long, narrow surface with a round tip at one of the narrow edges of the contact surface. In yet other words, the tip may be configured on a distal portion of the contact surface.

The blade-guiding channel may extend centrally, in an extension of the tip, in the metal electrode. The stiffening ribs may be arranged in a principal number transverse to the longitudinal extension of the metal electrode. A distance between the stiffening ribs may be substantially constant.

In a region of the round tip, a single one of the reinforcing ribs may be substantially aligned in the direction of extension of the blade-guiding channel.

In a further aspect, the stiffening ribs between the blade-guiding channel and the contact surface may be configured as substantially triangular angle pieces.

In other words, one edge of the stiffening ribs may be arranged at approximately a 45° angle to the contact surface.

Such an orientation or alignment of the stiffening ribs on/in the metal electrode allows bending forces to be absorbed in different spatial directions.

In a further aspect, the support portion may be surrounded by a plastic sheath.

In other words, on the side facing away from the clearance of the component, i.e. toward the outside, the gap may be configured with the plastic sheath, which covers the metal electrode. The plastic sheath acts as an electrical and/or thermal insulator and protects the porous support structure from punctual force attacks, since the plastic sheath introduces the applied force into the support structure in a distributed manner. Optionally, the plastic sheath may be configured with a predetermined elasticity, which additionally protects the support structure and thus the branch and the jaw part.

In a further aspect, the plastic sheath may be configured from an overmolding material that engages, at least in portions, with the porous support structure.

In other words, the plastic sheath may engage/penetrate the pores of the porous support structure and an interlocking/form-fitting between the plastic sheath and the porous support structure may be achieved. The plastic sheath may penetrate the porous support structure completely or superficially.

In yet other words, the plastic material may fill at least a portion of the porous volume of the support structure.

By configuring the plastic sheath in this way, mass accumulation during overmolding and the resulting component distortion may be avoided/prevented. Furthermore, a good, practically inseparable bond between the plastic sheath and the metal electrode may be created by the plastic sheath clawing into the porous support structure. In addition, an additional joining step between the metal electrode and the plastic sheath can be dispensed with during overmolding.

Furthermore, any (near-surface) defects in the porous support structure may be compensated for by the plastic of the plastic sheath.

In a further aspect, the porous support structure may be configured as a lattice/grid.

In other words, the porous support structure may be made up of three-dimensionally periodically arranged grid and cell structures. Common grid cells are body-centered cubic cells, face-centered cubic cells, simple cubic cells, or space trusses. Other possible grid types/lattice-grid structures are the part graph lattice, the volume graph lattice, the 3D conformal structure lattice, the unit graph lattice, the quad graph lattice, or the Gnd graph lattice.

Such grid structures may be produced in different porosities using additive manufacturing methods. A grid may absorb forces in different spatial directions and thus contribute to stiffening the jaw part or the metal electrode without significantly increasing the mass. The type of grid may be adapted/selected depending on the type and size of the jaw part. It is also conceivable to adapt/select the type of grid depending on the location on the metal electrode or a load situation.

In a further aspect, the porous support structure may be configured as a sponge structure or a bionic structure. A bionic structure is a structure that is based on geometries found in nature or biology.

In other words, the porous support structure may be configured, for example, as a honeycomb structure or as a load-oriented geometry. In yet other words, the porosity of the porous support structure may be configured differently/load-oriented at different points of the metal electrode. It is also conceivable that the dimensions of the rod structures may be changed or configured differently depending on the load situation/location on the metal electrode. For example, a load-oriented truss structure may be configured from the rod structures.

In yet other words, the contact surface of the metal electrode or the metal electrode may have high-load and low-load areas and the high-load areas may be reinforced with respect to the low-load areas.

Such a configuration may further reduce the thermally relevant mass of the metal electrode.

In a further aspect, hinge elements of the jaw part may be configured in one piece of material with the metal electrode.

In a further aspect, power-transmission elements of the HF instrument may be configured in one piece of material with the metal electrode.

In a further aspect, power transmission elements of the HF instrument may be configured in one piece of material with the metal electrode.

According to the disclosure, it is thus provided to provide a comparatively thin closed (non-porous/non-grid-like) contact surface, for example consisting of a metal layer or a metal platelet, which has an unstable (easily deformable) property against thermal and/or mechanical stress and to support (stiffen) it via an open (porous/grid-like) support structure (on the rear side/side facing away from the patient tissue), which has a stable (difficult to deform) property against thermal and/or mechanical stress, or, in other words, is stiff/stiffer than the contact surface (or the layer/plate forming the contact surface).

Furthermore, the object is solved by the HF instrument, in particular a bipolar vessel sealing instrument, with at least one metal electrode which comprises a contact surface for contacting tissue, wherein the metal electrode is produced by a generative manufacturing method/an additive manufacturing method/3D printing.

### BRIEF DESCRIPTION OF THE DRAWINGS

FIG. 1 shows an HF instrument according to the disclosure;

FIG. 2 shows a jaw part of the HF instrument according to the disclosure with two branches;

FIG. 3 shows a metal electrode of the branch of the jaw part without a porous support structure;

FIG. 4 shows the metal electrode of the branch of the jaw part with the porous support structure; and

FIG. 5 shows a cross-section of only the branch of the jaw part.

### DETAILED DESCRIPTION

Embodiments of the present disclosure are described below on the basis of the accompanying Figures.

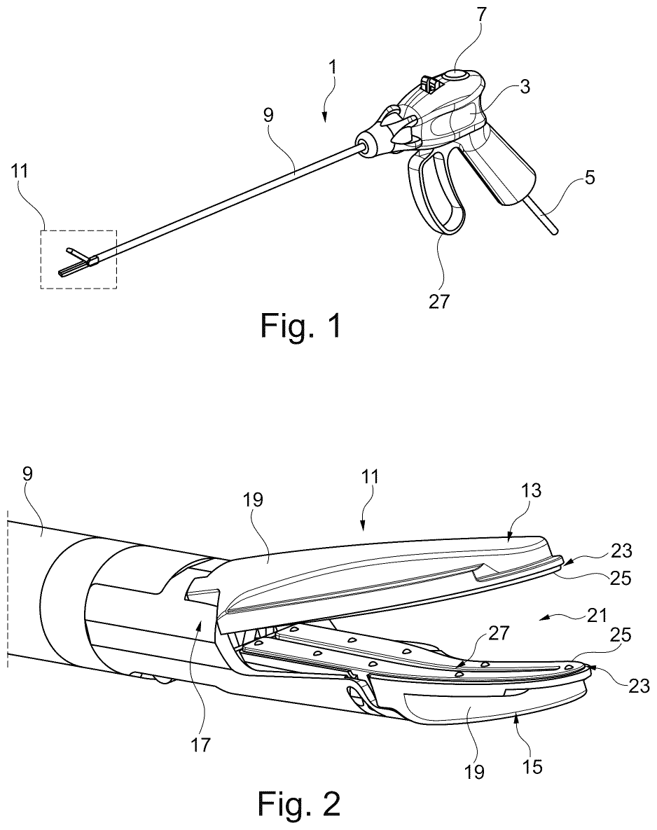

FIG. 1 shows an HF instrument according to the disclosure in the form of a bipolar vessel sealing instrument 1. The vessel sealing instrument 1 includes an actuation or handle portion 3 at a proximal end. The actuation or handle portion 3 is provided and configured to be gripped by an operator/surgeon and forms a substantially pistol-shaped handle portion. A cable 5 is also configured on the actuation or handle portion 3, which connects the vessel sealing instrument 1 to a high-frequency generator (not shown). Furthermore, the actuation or handle portion 3 contains a button 7, which may be used to request an energy supply from the high-frequency generator by actuating.

A shaft 9 is connected distally to the actuation or handle portion 3. The shaft 9 has a substantially rod-shaped or tubular geometry. The jaw part 11 is configured at a distal end of the shaft 9 and thus at a distal end of the vessel sealing instrument 1. The jaw part 11 is provided and configured to act as a cutting and sealing portion with which the operator may make cuts in tissue portions and on vessels and then close or seal cut areas and/or vessels.

FIG. 2 shows an enlarged view of the jaw part 11 in an open state. The jaw part 11 includes a first branch 13 and a second branch 15, which are hinged together by a hinge 17. Both the first branch 13 and the second branch 15 contain an outward-facing plastic sheath 19.

In the open state of the jaw part 11, the clearance 21 of the jaw part 11 is configured between the two branches 13, 15. Both the first branch 13 and the second branch 15 further include a metal electrode 23. The metal electrode 23 includes a contact surface 25. The contact surface 25 of the first branch 13 and the contact surface 25 of the second branch 15 substantially delimit the clearance 21 as a flat surface. The contact surfaces 25 are provided and configured to contact the tissue. The first branch 13 and the second branch may be moved relative to each other around the hinge 17 via a handle (see 27 in FIG. 1). A respective blade-guiding channel 27 open toward the clearance 21 is configured in the contact surface 25 of the first branch 13 and in the contact surface 25 of the second branch 15. The blade-guiding channel 27 is provided and configured to guide a blade (not shown) in the axial direction, starting from the shaft 9 or starting from a blade element configured on the shaft 9. The axial direction is to be understood as a longitudinal direction of the shaft 9.

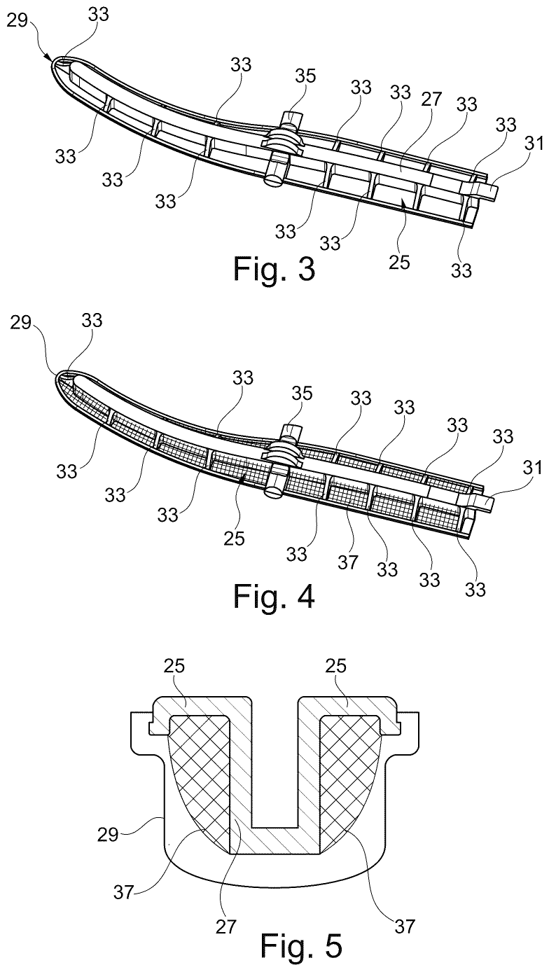

FIG. 3 shows the metal electrode 23 from a rear side or from a side facing away from the contact surface 25. In the embodiment shown in FIG. 3, a porous support structure is not shown for better illustration.

The metal electrode 23 has a substantially tongue-shaped geometry with a tip 29 configured at a distal end of the metal electrode 23. A power-transmission element 31 is configured at a proximal end of the metal electrode 23. The blade-guiding channel 27 extends between the tip 29 and the power-transmission element 31. The blade-guiding channel 27 has a groove-shaped geometry that is open toward the contact surface 25 and extends in a width direction of the metal electrode 23 centrally away from the contact surface 25 to the rear side. The metal electrode 23 furthermore includes stiffening ribs 33, which project vertically from the blade-guiding channel 27. The stiffening ribs 33 are configured as substantially triangular angle pieces between the blade-guiding channel 27 and the contact surface 25. The stiffening ribs 33 are arranged evenly distributed over the longitudinal extension of the metal electrode 23. A stiffening rib 33 is furthermore configured on the tip 29, which extends substantially in the longitudinal direction of the blade-guiding channel 27.

The metal electrode 23 further includes a hinge bolt 35, which extends in the width direction of the metal electrode 23. The hinge bolt 35 has a substantially round cross-sectional area and is part of the hinge 17.

The metal electrode 23 is configured in one piece of material. In other words, the contact surface 25, the blade-guiding channel 27, the power-transmission element 31, the stiffening ribs 33, and the hinge bolt 35 are configured in one piece of material.

FIG. 4 shows the metal electrode 23 from FIG. 3 with a porous support structure in the form of a grid 37. The grid 37 is configured between the contact surface 25, the blade-guiding channel 27, and the stiffening ribs 33. The grid 37 is configured in one piece of material with the metal electrode 23. The grid 37 is a three-dimensional grid structure. The grid 37 forms a support portion 39 with the stiffening ribs 33. This support portion 39 substantially forms the rear side of the metal electrode 23 and increases the rigidity of the metal electrode 23 or the associated branch 13, 15. The grid 37 is substantially configured in spaces or space portions delimited by the stiffening ribs 33, the blade-guiding channel 27, and the contact surface 25. Starting from the contact surface 25, the grid 37 may extend at the rear side of the metal electrode 23 substantially as far as the blade-guiding channel 27.

In an alternative embodiment, the support portion 39 may be configured only from the grid 37. In other words, the metal electrode 23 may be configured without the stiffening ribs 33.

The grid 37 is an additively manufactured support structure. In other words, the grid 37 is configured using generative manufacturing methods/additive manufacturing methods/3D printing from metal. Preferably, the entire metal electrode 23 is additively manufactured. However, embodiments are also conceivable in which the grid 37 gets or is additively formed on a base body of the metal electrode 23.

The grid 37 is shown here as a cuboid grid. Of course, other grid shapes, sponge shapes, or load-adapted bionic structures, for example in the form of honeycombs, are also conceivable. FIG. 5 shows a cross-section through one of the branches 13, 15 of the jaw part 11. The contact surface 25 forms substantially a T-shape with the U-shaped blade-guiding channel 27. The grid 37 is configured between the blade-guiding channel 27 and the contact surface 25. The plastic sheath 19 is configured as an overmolding material. This means that the plastic sheath 19 engages in pores of the grid 37 and is thus form-fittingly connected to the metal electrode 23. In other words, the metal electrode 23 forms a branch 13, 15 integrally with the plastic sheath 19. The plastic sheath 19 may only penetrate the grid 37 superficially or may penetrate the grid 37 almost completely.

### LIST OF REFERENCE SIGNS

1 vessel sealing instrument
3. actuation or handle portion
5 cable
7 button
9 shaft
11 jaw part
13 first branch
15 second branch
17 hinge
19 plastic sheath
21 clearance
23 metal electrode
25 contact surface
27 blade-guiding channel
29 tip
31 power-transmission element
33 stiffening ribs
35 hinge bolt
37 grid
39 support portion

1-14. (canceled)

15. An HF instrument comprising:
a branch having a jaw part; and
a metal electrode configured in the jaw part,
the metal electrode comprising a contact surface that is solidly configured for contacting tissue,
the contact surface comprising a flat portion and facing a clearance space of the jaw part, and
the metal electrode further comprising a support portion facing away from the contact surface, the support portion being or comprising a porous support structure.

16. The HF instrument according to claim 15, wherein the metal electrode is monolithically configured with the support portion.

17. The HF instrument according to claim 15, wherein the metal electrode comprises a blade-guiding channel for guiding a blade, the blade-guiding channel being open toward the contact surface and extending in a longitudinal direction of the metal electrode.

18. The HF instrument according to claim 17, wherein:
the blade-guiding channel and the contact surface form a T-shape with each other; and
the porous support structure is configured in a space or portion delimited by the contact surface and the blade-guiding channel.

19. The HF instrument according to claim 17, wherein the support portion comprises stiffening ribs.

20. The HF instrument according to claim 19, wherein:
the contact surface is tongue-shaped, and
the stiffening ribs are aligned normal to the blade-guiding channel.

21. The HF instrument according to claim 20, wherein the stiffening ribs are distributed uniformly over a longitudinal extension of the metal electrode.

22. The HF instrument according to claim 19, wherein the stiffening ribs are between the blade-guiding channel and the contact surface and are configured as triangular angle pieces.

23. The HF instrument according to claim 15, wherein the support portion is surrounded by a plastic sheath.

24. The HF instrument according to claim 23, wherein the plastic sheath is made from an overmolding material that engages in the porous support structure.

25. The HF instrument according to claim 24, wherein the porous support structure forms a grid.

26. The HF instrument according to claim 15, wherein the porous support structure forms a sponge structure or a bionic structure.

27. The HF instrument according to claim 15, wherein the metal electrode is configured in one piece of material.

28. The HF instrument according to claim 15, wherein the porous support structure is a rigid porous support structure.

29. The HF instrument according to claim 15, wherein the HF instrument has a scissor or pincer configuration.

30. The HF instrument according to claim 15, wherein the HF instrument has a laparoscope configuration.

31. An HF instrument comprising:
a branch having a jaw part; and
a metal electrode configured in the jaw part,
the metal electrode comprising a contact surface for contacting tissue,
the contact surface comprising a flat portion and facing a clearance space of the jaw part, and
the metal electrode further comprising a grid structure.

32. The HF instrument according to claim 31, wherein the metal electrode is configured in one piece of material.

33. The HF instrument according to claim 31, wherein the HF instrument has a scissor or pincer configuration.

34. The HF instrument according to claim 31, wherein the HF instrument has a laparoscope configuration.
